(19) United States
(12) Patent Application Publication
(10) Pub. No.: US 2003/0224069 A1
(43) Pub. Date: Dec. 4, 2003

(54) Triterpene glycosides for hormonal therapy

(76) Inventor: Ven Subbiah, Greenville, NC (US)

Correspondence Address: MYERS BIGEL SIBLEY & SAJOVEC, PO BOX 37428, RALEIGH, NC, 27627, US

(21) Appl. No.: 10/162,163

(22) Filed: Jun. 3, 2002

(51) Int. Cl. A61K 35/78

(52) U.S. Cl. 424/725; 424/764

(57) ABSTRACT

Triterpene glycosides are provided in using a multi-step process. The plant of the Cimicifuga genus (e.g., the root portion) is contacted with an organic solvent to extract certain components, including triterpene glycosides, from that plant. The resulting extracted portion is subjected to a reduction step to convert some of the triterpene glycosides to compounds such as 27-deoxyactein. Optionally, compounds such as 27-deoxyactein then are isolated, preferably by chromatographic techniques. The present invention also provides a therapeutic composition comprising a therapeutically effective amount of the triterpene glycosides provided according to the above method.

### FIELD OF THE INVENTION

The present invention relates to compounds derived from black cohosh plants that are useful for promoting wellness and good health, and particularly for treating hormonal imbalance in women.

### BACKGROUND OF THE INVENTION

As women age, various health issues occur which are often associated with hormone imbalance caused by menopause or post menstrual stress (PMS). Menopause, which typically occurs in women during middle age, is often described as ovarian shutdown. Menopause is typically associated with a significant decrease in circulatory estrogens. Such imbalance causes certain chemical reactions to occur, and resulting symptoms include headaches, cramping, nausea, inflammation, increase agitation, anxiety, tension, restlessness, decreased digestive tract activity, moodiness, and severe mood swings. Known treatments primarily focus on administration of estrogens, so-called “estrogen replacement therapy” to women.

It would be desirable to have various homeopathic remedies for treating such symptoms as an alternative to estrogen replacement therapy. Various homeopathic methods for treating the symptoms associated with menopause are known. For instance, there are several herbal remedies that are used to alleviate menopausal symptoms. One example of a commercially available homeopathic remedy for treatment of menopausal symptoms is Hyland's Menopause Tablets, which contains a specific combination of Amyl Nitrosum, Sanguinaria Canadensis and Lachesis Mutus in a lactose base. Other known homeopathic products useful in treating various conditions are described in the Homeopathic Pharmacopeia of the United States (HPUS).

One natural material that has been associated with treatment of hormonal imbalance in women is derived from portions of the black cohosh plant. The black cohosh plant (Cimicifuga racemosa) is a plant in the buttercup family, and part of the Cimicifuga plant genus. Black cohosh is native to eastern North America. Its rhizomes have been used to treat a variety of ailments such as diarrhea, sore throat, rheumatism, and particularly painful menstrual periods and menopausal disorders. See, for example Shao et al. J. Nat. Prod. 2000, 63, 905-910.

The black cohosh plant has a high level of triterpene glycosides, and further includes agylcones, isofalvones and aromatic acids. It is believed that such triterpene glycosides are the active components of black cohosh. See, U.S. Pat. No. 6,248,307 to Borneman et al. Triterpene glycosides components include 27-deoxyactein, 26-deoxycimicifugoside, actein, acetyl shengmanol xyloside, cimicifugoside (cimigenol-3-O-β-D-Xylopyranoside), cimiaceroside A, 12β-hydroxycimigenol-3-O-β-D-xylopyranoside, 12β-hydroxycimigenol-3-O-α-L-arabinopyranoside, 21-hydroxycimigenol-3-O-α-L-arabinopyranoside, 21-hydroxycimigenol-3-O-β-D-xylopyranoside, cimigenol-3-O-α-L-arabinopyranoside, 12β-acetoxycimigenol-3-O-α-L-arabinopyranoside, 24-acetylisodahurinol-3-O-β-D-xylopyranoside, 20(S),22(R),23(S),24(R)-16β:23;22;:25-diepoxy-12β-acetoxy-3β,23,24-trihydroxy-9,19-cycloanost-7-ene-3-O-β-D-xylopyranoside, 20(S),22(R),23(S),24(R)-16β:23;22;:25-diepoxy-12β-acetoxy-3β,23,24-trihydroxy-9,19-cycloanost-7-en-3-O-β-L-arabinopyranoside, and 20(S),22(R),23(S),24(R)-16β:23;22;:25-diepoxy-12β-acetoxy-3β,23,24-trihydroxy-9,19-cycloanostane-3-O-β-D-xylopyranoside. (Shao et al., p. 905). It has been demonstrated that black cohosh extract may exert its effect on menopausal women by reducing the serum concentration of the pituitary hormone luteinizing hormone (LH). See, Duker, E. M. Et al., Effects of black Cohosh Extract from Cimicifuga Racemosa on Gonadotropin Release in Menopausal Women and Ovariectomized Rats, Planta Medica 57: 420-424.

Techniques for separating the triterpene glycosides from the black cohosh plant have been suggested. U.S. Pat. No. 6,267,994 to Nesselhut et al. suggests comminuting the rhizomes of the black cohosh plant, macerating in isopropyl alcohol and separating the alcohol from the macerated rhizome. Additionally, a powdered extract of black cohosh is available as CimiPure® from Madis Botanicals. Using black cohosh to provide a homeopathic remedy is, however, limited by the known extraction techniques, many of which are economically not feasible on a large production scale. Thus, there is a need for a black cohosh extraction method that is commercially feasible can be used to isolate on or more therapeutic glycosides, and can provide a homeopathic remedy for treatment of hormonal imbalance conditions.

It would be desirable to provide an efficient and effective manner or method for providing specific types of triterpene glycosides at high yield in relatively pure form.

### SUMMARY OF THE INVENTION

The present invention relates to separating certain specific triterpene glycosides from a portion of a plant of the Cimicifuga genus such as the black cohosh plant. One specific triterpene glycoside is 27-deoxyactein. The process comprises contacting at least a portion of the plant with an organic solvent (e.g., methylene chloride) to provide a mixture (e.g., a slurry) of the plant portion and the organic solvent. The mixture most preferably is subjected to conditions sufficient to separate a solvent insoluble plant portion from an extracted plant portion within the solvent (e.g., filtration). The extracted plant portion, preferably in solid form (e.g., due to removal of the solvent from the extract), is subjected to a reduction step (e.g., using sodium borohydride in a suitable solvent, such as tetrahydrofuran) to convert at least a portion of the triterpene glycosides that were extracted by the solvent to compounds such as 27-deoxyactein. The portion of the resulting mixture containing the desired components, including compounds such 27-deoxyactein, is separated from that mixture (e.g., using phase separation techniques). The reduced extracted components from within the separated phase then are provided. Preferably, the triterpene glycosides are collected to a relatively high yield. Optionally, certain specific triterpene glycosides, such as 27-deoxyactein, then are separated, and preferably is isolated, most preferably using chromatographic techniques. Removal of solvent from the predetermined chromatographic fractions yields a powdered material in the form of triterpene glucrosides, preferably including high levels of 27-deoxyactein at very high purity.

The triterpene glycosides, which most preferably is in powder form, can be formulated into a desirable form for use. For example, the powder can be combined with other suitable components for ingestion or administration, such as a capsule or tablet.

The triterpene glycosides can be used in a convenient form to, or ingested by, a human patient or subject for the purposes of improving the overall wellness of a patient, and particularly a female patient or subject in need of hormone replacement therapy. Triterpene glycosides can be used to supplement a diet by indigestion on a daily basis alone or in combination with other natural or synthetic hormones to result in reduction of such ailments as headaches, cramping, nausea, inflammation, increase agitation, anxiety, tension, restlessness, decreased digestive tract activity, moodiness, and severe mood swings.

### DETAILED DESCRIPTION OF THE INVENTION

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

As summarized above, the present invention relates to a process of providing triterpene glycosides from black cohosh root. As such, those compounds are obtained from a natural source, and are in a form suitable for treating hormone imbalance. Such treatment of hormonal imbalance can include the ingestion of a beneficially or therapeutically effective amount of those compounds as a dietary supplement on a specific regiment, (e.g., daily). The beneficially or therapeutically effective amount can be taken in combination with other known natural (herb) compositions or synthetic compositions useful in the treatment of hormone imbalance.

A dietary supplement is defined under the Dietary Supplement Health and Education Act of 1994 (“DSHEA”). A dietary supplement is a product (other than tobacco) intended to supplement the diet, and contains one or more of the following dietary ingredients: a vitamin; a mineral; an herb or other botanical; an amino acid; a dietary substance for use by a human to supplement the diet by increasing the total dietary intake; or a concentrate, metabolite, constituent, extract, or combination of any of those ingredients. The product is intended for ingestion in tablet, capsule, powder, softgel, gelcap, or liquid form. As defined by DSHEA, a dietary supplement is not represented for use as a conventional food or as a sole item of a meal or of the diet.

As used herein and in the claims, the terms “beneficially effective amount” or “therapeutically effective amount” mean an amount of material or composition sufficient to significantly induce a positive modification in the condition to be treated, but low enough to avoid serious side effects (at a reasonable benefit/risk ratio) within the scope of sound medical judgment. The beneficially or therapeutically effective amount of the material or composition can vary depending upon factors such as the particular condition being treated, the overall metabolism of the person, the age and physical condition of the person, the severity of the condition, the overall metabolism of that person, the duration of the treatment, the nature of concurrent therapy, the specific composition employed, and like factors within the knowledge and expertise of the medical community.

The triterpene glycosides can be provided by using the process steps and conditions of the present invention. The starting point for providing the triterpene glycosides involves contacting the black cohosh plant (or portion thereof) with an organic solvent. The black cohosh plant can be in a form that has not been previously subjected to extraction with any type of solvent or it can be in a form that has undergone some form of chemical or physical processing. The plant or any portion thereof can also be pre-treated with agents capable of enhancing the effectiveness of extraction and isolation of compounds therefrom. Most preferably, the portion of the plant that is used comprises a high concentration of the components required to provide the desired product. Typically the root or rhizomes are separated from the rest of the plant. Preferably, the portion of the plant that is used comprises the root. It is recognized that black cohosh (Cimicifuga racemosa) is part of the Cimicifuga plant genus which includes Cimicifuga simplex and Cimicifuga foetida. The process of the present invention can be practiced on other species of the Cimicifuga plant genus.

A portion of the black cohosh plant is contacted with an organic solvent to provide a mixture, preferably in the form of a slurry, of the plant portion and the organic solvent. The organic solvent is used to extract relevant components from within the black cohosh plant. Those components can be extracted from the black cohosh plant using a solvent that is in the form of a liquid, or the components can be extracted using a suitable solvent under supercritical extraction conditions or supercritical-type conditions. The organic solvent can be anhydrous or nonanhydrous in nature. Suitable organic solvents include compounds containing at least one carbon atom, and include alkanes (e.g., pentanes, heptanes, hexanes, octanes and cyclohexane), alcohols (e.g., methanol, ethanol, propanols, butanols, pentanols and other types of lower alcohols), ethers (e.g, diethyl ether), petroleum ethers, halocarbons and halogenated hydrocarbons (e.g., chloroform and methylene chloride), carbonyl-containing compounds (e.g., acetone and methyl ethyl ketone), and other organic compounds, such as tetrahydrofuran, toluene, and ethyl acetate. A preferred solvent is methylene chloride. Solvents typically used for the extraction in the food and dietary supplement industries are particularly preferred, and food grade solvents are of particular interest. For example, food grade denatured ethanol is a particularly preferred solvent. For purposes of the present invention, an organic solvent is a liquid solvent comprised primarily of organic liquid, or a supercritical fluid comprising at least one compound containing at least one carbon atom, and can be essentially pure organic solvent. The extraction also can be carried out under the supercritical conditions of the organic solvent (i.e., under conditions of temperature and pressure that define supercritical conditions or supercritical-type conditions). Supercritical fluids can include carbon dioxide, compound such as the alkanes (e.g., including methane, ethane, butane and pentane), halocarbons, halogenated hydrocarbons, on combinations thereof. Supercritcal fluid and supercritical-type extraction materials, equipment, procedures and conditions suitable for extracting components of plant materials are well known. See, for example, U.S. Pat. No. 4,153,063 to Roselius; U.S. Pat. No. 4,506,682 to Muller; U.S. Pat. No. 4,714,617 to Gahrs; U.S. Pat. No. 5,018,540 to Grubbs et al.; U.S. Pat. No. 5,435,325 to Clapp et al.; U.S. Pat. No. 5,554,382 to Castor; U.S. Pat. No. 5,639,431 to Castor and U.S. Pat. No. 6,095,134 to Sievers et al.; U.S. Pat. No. 6,111,108 to Lopez-Avila and U.S. Pat. No. 6,291,241 to Castor et al.; the disclosures of which are incorporated herein by reference in their entireties.

The solvent mixture, particularly when water is a component thereof, can include pH buffers, pH adjusters, organic and inorganic salts, sugars, surfactants, agents to facilitate extraction, or other additives.

The extraction preferably is performed at an optimized solvent to black cohosh plant ratio, namely at a ratio wherein a relatively large amount of extract is extracted from the black cohosh plant using a balance of the optimum and minimum amount of solvent. Typically, the lower amount of solvent used, the more concentrated the extract within the solvent. The ratio of organic solvent to black cohosh plant for the extraction preferably is about 1 to about 10, often is about 2 to about 5, and more often is about 2 to 3, on a weight basis.

The manner by which the extraction is carried out can vary. The extraction can be carried out using a liquid organic solvent in a batch or continuous manner. Suitable equipment used to carry out the extraction will be readily apparent to those skilled in the art of extraction of natural materials, such as vegetables, fruits, herbs, and the like. Exemplary extraction equipment is commercially available and is used throughout the food and dietary supplement industries. Exemplary types of extraction equipment can include high shear blenders, food processing mills, counter current extractors, mixing drums, percolators, static mixers, and the like. Preferred extraction equipment is suitably equipped to provide the desired heating of the mixture being extracted. For example, extractors can be equipped with suitable heating jackets. Preferably, the mixture that is being subjected to extraction conditions is agitated. That is, the mixture of solvent and black cohosh plant experiences some type of movement during the extraction period, and that movement is supplied in order to facilitate extraction of the desired components from the black cohosh plant by the solvent. Such agitation can be provided by high shear mixing, stirring, squeezing, shaking, or other like types of movement. Suitable extraction techniques and apparatus are described in U.S. Pat. No. 5,234,008 to Fagg and U.S. Pat. No. 5,360,022 to Newton, the disclosures of which are incorporated herein by reference in their entireties.

The solvent and extracted components therein most preferably are separated from the solvent insoluble portion of the black cohosh plant. As such, black cohosh “pulp” is separated from the liquid portion of the processed mixture, and typically is discarded. It is desirable to remove as much of the dispersed and insoluble portions from the mixture as possible, however, it is not strictly necessary to remove virtually all of the dispersed and insoluble portions from the solvent containing the extracted components. Techniques for such separation will be readily apparent to those having skill in the art of slurry handling, and in liquid extraction of vegetables, fruits, herbs and other plant materials. Suitable techniques involve the use of filters, screens, centrifuges, presses, screw presses, rotating disk presses, converging belts, and the like. As such, significantly high amounts of the desired black cohosh plant extract and solvent are obtained. Then, the portion of the plant extract is isolated, typically as a solid, and preferably in powder form.

The conditions under which the solvent separation is performed can vary. Extractions using liquid organic solvents typically are carried out under conditions of atmospheric pressure, or under slight vacuum conditions (e.g., about 3 to about 10 inches of water column vacuum). Conditions of temperature can be less than, greater than, or equal to, ambient temperature. Typical temperatures (depending upon the characteristics of the solvent or co-solvent mixture) can range from about 5° C. to about 55° C. often about 10° C. to about 60° C., and frequently about 15° C. to about 65° C. It is most preferred that the extraction be carried out while the mixture of black cohosh plant and solvent is maintained at temperatures above ambient temperature. As such, preferred extraction conditions involve heating that mixture. The heating is often performed at a temperature of about 5° C. to about 25° C. less than the boiling point of the solvent in the black cohosh plant/solvent mixture. Known rotary/evaporation equipment and techniques are suitable for providing such solvent separation from the extract. Other suitable techniques for removing solvent from extract to provide the extract in a solid, powdered form, include spray drying and freeze drying equipment and methodologies. See, for example, U.S. Pat. No. 5,005,593 to Fagg and U.S. Pat. No. 3,316,919 to Green.

The black cohosh plant extract, which most preferably is in a solid (e.g., powder) form, is subjected to conditions sufficient to reduce the extracted solvent insoluble plant portion utilizing a medium sufficient to reduce the extracted plant portion, namely a reducing agent and a reducing solvent to provide reduced triterpene glycoside components of the extracted plant portion. For purposes of this invention reduction and conditions sufficient involve a chemical reaction of components of the extracts to provide the desired triterpene glycosides including 27-deoxyactein at high yields and/or high priority. Suitable reducing agents include sodium borohydride, lithium aluminum diborane and sodium bis(2-methoxyethoxy) aluminum dihydride. Suitable reducing solvents (i.e., solvents that are used to provide a medium for the reduction of the extract using the reducing agent) are organic solvents, and include alkanes (e.g., pentanes, heptanes, hexanes, octanes and cyclohexane), alcohols (e.g., methanol, ethanol, propanols, butanols, pentanols and other types of lower alcohols), ethers (e.g, diethyl ether), petroleum ethers, halocarbons and halogenated hydrocarbons (e.g., chloroform and methylene chloride), carbonyl-containing compounds (e.g., acetone and methyl ethyl ketone), and other organic compounds, such as tetrahydrofuran, toluene, and ethyl acetate. A preferred solvent is tetrahydroflran. The amount of reducing agent relative to the extract can vary; but typically is about 1:3 to about 1:6, preferably about 1:4 to about 1:5, on a weight basis. The amount of reducing solvent relative to extract can vary; but typically is about 10:1 to about 50:1, preferably about 20:1 to about 40:1, on a weight basis.

The reduced extracted plant portion and reducing solvent is subjected to fractionation. Typically, such a fractionation or separation is conducted through the use of phase separation using a suitable solvent that different from the reducing solvent. Preferably, that solvent is essentially immiscible with the reducing solvent. For example, when the reducing solvent is tetrahydrofuran, phase separation can be carried out using ethyl acetate or a lower alkyl alcohol (e.g., methanol or ethanol) to provide a fractionated mixture of reduced extracted plant extract and solvent. A preferred fractionation technique is Kupchan fractionation such as described in U.S. Pat. Nos. 3,969,369, 4,005,108, and 4,164,584 to Kupchan et al., the disclosures of which are incorporated herein in their entireties. As a result, the desired components of the reduced plant extract are provided from within the reducing solvent to within that other solvent.

The reduced extract within the solvent used for fractionation then can be separated from that solvent. Preferably, that solvent is removed by evaporation, using known techniques, such as rotary/evaporation techniques. Other suitable techniques for removing solvent from extract to provide the extract in a solid, powdered form, include spray drying and freeze drying equipment and methodologies. As such, mixtures incorporating triterpene glycosides are provided. Typically, such mixtures have triterpene glycoside contents of greater than about 10 percent, often greater than about 15 percent, and preferably greater than about 20 percent, by weight.

The desired components within the resulting reduced extract are separated from that extract. Optionally, specific components can be isolated, often in a very pure form. For example, 27-deoxyactein is isolated from the reduced plant extract using suitable techniques. Other components include 26-deoxycimicifugoside, actein, acetyl shengmanol xyloside, cimicifugoside (cimigenol-3-O-β-D-Xylopyranoside), cimiaceroside A, 12β-hydroxycimigenol-3-O-β-D-xylopyranoside, 12β-hydroxycimigenol-3-O-α-L-arabinopyranoside, 21-hydroxycimigenol-3-O-α-L-arabinopyranoside, 21-hydroxycimigenol-3-O-β-D-xylopyranoside, cimigenol-3-O-α-L-arabinopyranoside, 12β-acetoxycimigenol-3-O-α-L-arabinopyranoside, 24-acetylisodahurinol-3-O-β-D-xylopyranoside, 20(S),22(R),23(S),24(R)-16β:23;22;:25-diepoxy-12β-acetoxy-3β,23,24-trihydroxy-9,19-cycloanost-7-ene-3-O-β-D-xylopyranoside, 20(S),22(R),23(S),24(R)-16β:23;22;:25-diepoxy-12β-acetoxy-3β,23,24-trihydroxy-9,19-cycloanost-7-en-3-O-α-L-arabinopyranoside, and 20(S),22(R),23(S),24(R)-16β:23;22;:25-diepoxy-12β-acetoxy-3β,23,24-trihydroxy-9,19-cycloanostane-3-O-β-D-xylopyranoside.

Chromatographic methodologies provide one suitable technique, and representative chromatographic methodologies include flash column chromatography, vacuum liquid chromatography, and combinations thereof. The degree to which specific components of the triterpene glycoside mixture can be separated chromatographically can vary. For example, powdered reduced extract can be dissolved in a suitable elution solvent or solvent mixture, the resulting mixture passed through a chromatographic column, and fractions can be collected. Those fractions can be analyzed for the presence of 27-deoxyactein or other components, and desired fractions can be collected. Desired collected fractions can have elution solvent removed therefrom, and then subjected to further chromatographic treatment one or more times, in order to provide fractions that comprise, for example, 27-deoxyactein at high purity.

After the optional chromatographic separation, the desired components are separated from the elution solvents. Typically, the solvents are evaporated using known techniques, such as rotary/evaporation techniques. Other suitable techniques for removing solvent from desired components to provide those components in a solid, powdered form, include spray drying and freeze drying equipment and methodologies. As such, the desired components are isolated. By “isolation” it is meant that the isolated triterpene glycoside component is in substantially pure form and is at least 95 percent, often at least 98 percent, and sometimes at least 99 percent pure, by weight. Additionally, an enriched mixture can be obtained, having a percent by weight of triterpene glycosides of greater than 10 percent, often greater than 15 percent by weight and sometimes greater than 20 percent by weight triterpene glycosides. Typically, the range is from 15 to 20 percent by weight. Methods for determining the content of triterpene glycosides, such as 27-deoxyactein, in collected samples are of the type generally described in Kusana et al., Chem. Pharm. Bull. (Tokyo), 43: 771-776 (1996).

The triterpene glycosides described above may be formulated for administration in a pharmaceutical carrier in accordance with known techniques. See, e.g., Remington, The Science And Practice of pharmacy (9^{th} Ed. 1995). In the manufacture of a pharmaceutical formulation according to the invention, the extract is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5 percent to 95 percent or 99 percent by weight of the extract.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated.

Formulations suitable for oral administration may be presented in discrete units, such as capsules, cachets, lozenges, or tables, each containing a predetermined amount of the black cohosh extract; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the isolated triterpene glycosides and a suitable carrier (which may contain one or more accessory ingredients as noted above). In general, the formulations of the invention are prepared by uniformly and intimately admixing the isolated triterpene glycosides with a liquid or finely divided solid carrier, or both, and then, if necessary, shaping the resulting mixture. For example, a tablet may be prepared by compressing or molding a powder or granules containing the isolated triterpene glycosides, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the isolated triterpene glycosides in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the isolated triterpene glycosides, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unitdose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described.

Formulations suitable for rectal administration are preferably presented as unit dose suppositories. These may be prepared by admixing the triterpene glycosides with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)).

The therapeutically effective dosage of any triterpene glycoside mixture, the use of which is in the scope of present invention, will vary somewhat from compound to compound, and patient to patient, and will depend upon factors such as the age and condition of the patient and the route of delivery. Such dosages can be determined in accordance with routine pharmacological procedures known to those skilled in the art.

As a general proposition, a preferred dosage from about 20 to 50 mg/kg will have therapeutic efficacy, with all weights being calculated based upon the weight of the total weight of the triterpene glycosides. Toxicity concerns at the higher level may restrict intravenous dosages to a lower level such as up to about 20 mg/kg, with all weights being calculated based upon the weight of the active material. A preferred dosage from about 20 mg/kg to about 50 mg/kg may be employed for oral administration. Typically, a preferred dosage from about 30 mg/kg to 60 mg/kg may be employed for intramuscular injection.

Particular dosing regimens will ultimately be determined through clinical trials and will depend on the judgment of the attending physician. The dosage regimens may be used alone or may be combined with existing techniques for treating various women's health issues. Specifically the present invention discloses various methods that may be used to treat women's health issues. These women's health issues include the terms “frailty” and “wellness.” More specifically, these terms as used herein include age-related decline in muscle mass and strength and weight. They also include loss of sense of balance, loss of bone strength and loss of resistance to disease. By improving frailty via hormonal replacement therapy a subject is able to have greater energy, better quality of sleep, greater ability to maintain balance and increased feelings of well-being. Such regiments may be continuous and uninterrupted, one or more times a week in a predetermined cycle, or pattern or on demand.

The present invention is explained in greater detail in the following Example. This Example is intended as illustrative of the invention and is not to be taken as limiting thereof.

### EXAMPLE

500 g of dried powder of black cohosh plant root was mixed with 1000 ml of methylene chloride. This mixture was stirred at room temperature overnight to form a slurry. The slurry was the insoluble pulp of the root and the extract portion soluble in methylene chloride. The slurry was filtered to provide an extract within methylene chloride, and then that extract was separated from the solvent via rotovaporization (at about 45° C. and about 49 inches Hg) to provide the extract in dry, powder form. 40 g of the dried extract then was dissolved in 1000 ml of tetrahydrofuran. To the extract dissolved of tetrahydrofliran was added 10 g of sodium borohydride in 98 percent pure powder form. The resulting mixture was stirred continuously for 1.5 hours to reduce the triterpene glycoside components to obtain increased amounts of 27-deoxyactein within the solvent. The solution of reduced extract components in tetrahydrofuran then was phase separated using ethyl acetate, using conventional phase separation techniques. Then, the separated upper layer phase (i.e, the ethyl acetate phase) containing 27-deoxyactein was removed via rotovaporization (at about 45° C. and about 49 inches Hg) to provide a dried powder. That powder contained triterpene glycosides, including 27-deoxyactein.

Using silica gel chromatography (about 2 g of extract and about 8 liters of elution solvent in the form of 5 parts heptane, 5 parts ethyl acetate and 0.5 parts methanol, by weight; followed by C-18 reversed phase chromatography (selected fractions collected in elution solvent of 40 parts water and 60 parts methanol, by weight); 27-deoxyactein was obtained in elution solvent, and purified further by collection of selected fractions. Determination of the selection of the fractions used for the C-18 chromatographic separation was carried out by collecting 500 ml fractions, elution solvent was removed using rotovaporization (at about 45° C. and about 49 inches Hg) to provide the desired composition in powder form, and thin layer chromatography was performed on a very small amount of the resulting powdered sample, using a solvent system of 8.5 parts chloroform, 1.5 parts methanol and 0.1 parts acetic acid, by weight. For the desired fractions collected during C-18 chromatographic separation, the elution solvent was removed using rotovaporization (at about 45° C. and about 49 inches Hg) to provide the desired fractions in powder form, and then thin layer chromatography was performed on a very small amount of the resulting powdered samples, using a solvent system of 8.5 parts chloroform, 1.5 parts methanol and 0.1 parts acetic acid, by weight. The resulting solid material (i.e., 27-deoxyactein) that was collected from the desired fractions was isolated and was 95 weight percent pure. The purity was determined on a high pressure liquid chromatography system (ire., Agilent 1100 LC with a Sedex 55 ELSD attached to a HP 3396A integrator) using a water/formic acid gradient.

In the specification, there has been disclosed typical preferred embodiments of the invention and, although specific terms are employed, they are used in a generic and descriptive sense only and not for purposes of limitation of the scope of the invention being set forth in the following claims.

That which is claimed:

1. A process of providing at least one triterpene glycoside from at least a portion of a plant of the Cimicifuga genus, the process comprising:
(a) contacting at least a portion of the plant with an organic solvent to provide a mixture of the plant portion and the organic solvent;
(b) subjecting the mixture to conditions sufficient to separate a solvent insoluble plant portion from an extracted plant portion within the solvent;
(c) subjecting the extracted plant portion to conditions sufficient to reduce the extracted plant portion; and
(d) separating triterpene glycoside from the reduced extracted plant portion.

2. The process of claim 1, whereby the plant of the Cimicifuga genus is black cohosh.

3. The process of claim 2, whereby step (c) includes contacting the extracted plant portion with a medium sufficient to reduce the extracted plant portion.

4. The process of claim 3, whereby the medium sufficient to reduce the extracted plant portion includes a solvent and a reducing agent selected from the group consisting of sodium borohydride, lithium aluminum diborane and sodium bis (2-methoxyethoxy) aluminum dihydride.

5. The process of claim 4, whereby the solvent is tetrahydrofuran.

6. The process of claim 2, whereby step (b) includes subjecting the insoluble plant portion and extracted plant portion within the organic solvent to filtration to provide a mixture of extracted plant portion within organic solvent.

7. The process of claim 6, whereby organic solvent is separated from the extracted plant portion to provide extracted plant portion in solid form.

8. The process of claim 3, whereby step (d) includes phase separating the reduced extracted plant portion within the reducing medium using a solvent, removing the reduced plant portion from the solvent, and then separating triterpene glycosides using chromatographic techniques.

9. The process of claim 2, whereby the organic solvent of step (a) is in liquid form.

10. The process of claim 9, whereby the organic solvent of step (a) comprises methylene chloride.

11. The process of claim 2, whereby the portion of the black cohosh plant is the root.

12. A process of providing triterpene glycosides from at least a portion of a black cohosh plant, the process comprising:
(a) contacting at least a portion of the black cohosh plant with an organic solvent to provide a slurry of the plant portion and the organic solvent;
(b) subjecting the slurry to conditions sufficient to separate a solvent insoluble plant portion from an extracted plant portion within the solvent;
(c) providing the extracted plant portion in solid form;
(d) contacting the extracted plant portion with a reducing agent in a solvent under conditions sufficient to provide reduced triterpene glycoside components of the extracted plant portion; and
(e) separating the reduced extracted triterpene glycoside components from the reducing solvent using a solvent different from the reducing solvent to isolate those triterpene glycoside components.

13. The process of claim 12, whereby the reducing agent is selected from the group consisting of sodium borohydride, lithium aluminum diborane and sodium bis (2-methoxyethoxy) aluminum dihydride.

14. The process of claim 12, whereby the organic solvent of step (a) is liquid in form.

15. A method of treating hormonal imbalance conditions in a patient in need of treatment, the method comprising administering to the patient a therapeutically effective amount of triterpene glycosides isolated from a portion of a plant of the Cimicifuga genus.

16. The method according to claim 15, whereby the plant of the Cimicifuga genus is black cohosh.

17. The method according to claim 16, whereby the portion of the black cohosh plant is the root.

18. A process providing triterpene glycosides from at least a portion of a plant of the Cimicifuga genus, the process comprising:
(a) contacting at least a portion of the plant with an organic solvent to provide extracted plant portion within the solvent;
(b) subjecting the extracted plant portion to conditions sufficient to reduce the extracted plant portion; and
(c) separating triterpene glycosides from the reduced extracted plant portion.

19. The process of claim 18, whereby the plant of the Cimicifuga genus is black cohosh.

20. The process of claim 18, whereby step (b) includes contacting the extracted plant portion with a medium sufficient to reduce the extracted plant portion.

21. The process of claim 20, whereby the medium sufficient to reduce the extracted plant portion includes a solvent and a reducing agent selected from the group consisting of sodium borohydride, lithium aluminum diborane and sodium bis (2-methoxyethoxy) aluminum dihydride.

22. The process of claim 20, whereby the solvent is tetrahydrofuran.

23. The process of claim 20, whereby the portion of the black cohosh plant is the root.

24. A method of treating hormonal imbalance conditions in a patient in need of treatment, the method comprising administering to the patient a therapeutically effective amount of one or more triterpene glycosides isolated from at least a portion of a black cohosh plant.

25. The method according to claim 24, whereby the portion of the black cohosh plant is the root.

26. A pharmaceutical composition useful in the treatment of hormonal imbalance conditions, the composition comprising a therapeutically effective amount of one or more triterpene glycosides isolated from a portion of a black cohosh plant.

27. The pharmaceutical composition according to claim 26, wherein the therapeutically effective amount of enriched mixture of triterpene glycosides is at least 20 mg per kg of patient.

28. The pharmaceutical composition according to claim 26, wherein the therapeutically effective amount of enriched mixture of triterpene glycosides is from 20 to 50 mg per kg of patient administered orally.
